(19) United States
(12) Patent Application Publication
(10) Pub. No.: US 2024/0207373 A1
(43) Pub. Date: Jun. 27, 2024

(54) CYCLIC PEPTIDES FROM THE C-TERMINUS OF ACETYLCHOLINESTERASE FOR TREATMENT OF SKIN DISORDERS AND FOR COSMETICAL USE

(71) Applicant: NEURO-BIO LTD, Abingdon, Oxfordshire (GB)

(72) Inventor: Susan GREENFIELD, Abingdon (GB)

(21) Appl. No.: 17/909,730

(22) PCT Filed: Mar. 31, 2021
(86) PCT No.: PCT/GB2021/050802

§ 371 Date: Sep. 6, 2022

(30) Foreign Application Priority Data
Apr. 1, 2020 (GB) 2004803.9

(51) Int. Cl. A61K 38/46; A61K 8/66; A61Q 19/02

(52) U.S. Cl. CPC A61K 38/465; A61K 8/66; A61Q 19/02; C12Y 301/01007

(57) ABSTRACT

The invention relates to skin, and to novel compositions, therapies and methods for treating, preventing or ameliorating a variety of skin conditions. The invention also extends to cosmetics and pharmaceutical compositions, and methods of using them on skin to treat various conditions.

The invention relates to skin, and to novel compositions, therapies and methods for treating, preventing or ameliorating a variety of skin conditions. The invention also extends to cosmetics and pharmaceutical compositions, and methods of using them on skin to treat various conditions, such as eczema, psoriasis, melanoma, dermatitis, and acne, or as a skin whitening agent, or for reducing scarring.

The enzyme acetylcholinesterase (AChE) is expressed at different stages of development in various forms, all of which have the same catalytic enzymatic activity, but which have different molecular compositions. The ‘tailed’ (T-AChE—SEQ ID No: 1) is expressed at synapses and the inventors have previously identified two peptides that could be cleaved from the C-terminus of T-AChE, one being a 14 amino acid long peptide referred to as “T14” (SEQ ID No: 3), within the other which is a 30 amino acid long peptide known as “T30” (SEQ ID No: 2). The AChE C-terminal peptide “T14” has been identified as being the salient part of the AChE molecule responsible for its range of non-hydrolytic actions.

The synthetic analogue (i.e. “T14”), and subsequently the larger and more empirically tractable, and more potent amino acid sequence in which it is embedded (i.e. “T30”) display actions comparable to those reported for ‘non-cholinergic’ AChE, whereas the inert 15 amino acid long peptide within the T30 sequence (i.e. “T15”—SEQ ID No: 4) is without effect (Bond et al 2009 PLOS one Vol: 4 Issue: 3 e4846). The T14 peptide binds to an allosteric site on the α7 nicotinic-receptor, where, on its own, it has no effect. However, in the presence of a primary ligand, such as acetylcholine or dietary choline, T14 enhances the calcium influx induced by these primary agents. Excessive calcium can be taken up into the mitochondria where it compromises oxidative phosphorylation, and causes a leakage of electrons. Free radicals are consequently formed that then destabilize the cell membrane, and the cell then dies (Day & Greenfield 2004 Exp Brain Res 155: 500-508).

The epidermal layer of the skin is one of the few examples of a continuously renewing process in the mature adult. The cell cycle process is driven by activation of the alpha-7 receptor (Arreondo et al. 2002 J Cell Biol. 159(2):325-36), which is the target of T14 (Greenfield et al., 2004). In many common skin diseases, such as in psoriasis, the control mechanisms underlying the proliferation of keratinocytes (which comprise ˜90% of cells within the epidermis), associated with the renewal process, go awry. In such situations, the keratinocytes enter the alternative pathway of proliferation characterized by excessive growth rate, aberrant responses to growth factors, faulty differentiation, and increased migratory capacity.

Thus, there is a need to identify agents that reduce or prevent proliferation of keratinocytes that may be used in the treatment of skin conditions.

The inventors investigated the effects of cyclic peptides derived from the C-terminus of acetylcholinesterase on a keratinocyte cell line, and found that T30 (SEQ ID No: 2), a sequence comprising the T14 sequence (SEQ ID No: 3), stimulates intracellular calcium influx into the skin cells and induces cell proliferation. The inventors also surprisingly showed that a cyclic peptide derived from the C-terminus of acetylcholinesterase (known as “NBP-14”) inhibits T30-induced intracellular calcium influx into keratinocytes. Accordingly, the inventors believe that NBP-14 may be utilized as a therapeutic agent to treat, prevent or ameliorate skin conditions associated with cell proliferation, such as psoriasis and cancer.

Thus, in a first aspect of the invention, there is provided a cyclic polypeptide, derivative or analogue thereof comprising an amino acid sequence derived from the C-terminus of acetylcholinesterase (AChE), or a truncation thereof, for use in treating, preventing or ameliorating a skin disease.

In a second aspect, there is provided a method of treating, ameliorating or preventing a skin disease, the method comprising, administering, or having administered, to a subject in need of such treatment, a therapeutically effective amount of a cyclic polypeptide, derivative or analogue thereof comprising an amino acid sequence derived from the C-terminus of acetylcholinesterase (AChE), or a truncation thereof.

As described in the examples, the inventors performed intracellular calcium imaging and cell proliferation assays on the keratinocyte cell line, HaCaTs, a widely used and characterized model for human keratinocytes. The inventors have surprisingly shown that the linear peptide, T30, is able to induce an intracellular calcium influx in HaCaTs, which correlates with stimulation of cell proliferation. The inventors' further work has shown that, surprisingly, a cyclic peptide derived from the C-terminus of acetylcholinesterase (known as “NBP-14”) inhibits T30-induced intracellular calcium influx into keratinocytes, indicating that cyclic peptides derived from the C-terminus of acetylcholinesterase may be used in the treatment of skin conditions associated with aberrant keratinocyte proliferation.

The cyclic polypeptide, derivative or analogue thereof may target cells present in deep layers of the epidermis, for example the stratum basale, stratum spinsosum and/or stratum granlulosum. Preferably, the cyclic polypeptide, derivative or analogue thereof target cells in the stratum basale.

The skin condition which is treated is preferably a skin condition associated with abnormal keratinocyte proliferation. Preferably, the skin condition, which is treated, may be selected from a group consisting of: eczema, psoriasis, melanoma, dermatitis, and acne.

The cyclic polypeptide, derivative or analogue thereof may also be used as a skin whitening agent.

Thus, in a third aspect, there is provided the cyclic polypeptide, derivative or analogue thereof according to the first aspect, for use as a skin whitening agent.

Cyclic polypeptides are peptide chains whose N- and C-termini are themselves linked together with a peptide bond that forms a circular chain of amino acids.

The term “derivative or analogue thereof” can mean a polypeptide within which amino acid residues are replaced by residues (whether natural amino acids, non-natural amino acids or amino acid mimics) with similar side chains or peptide backbone properties. Additionally, the terminals of such peptides may be protected by N- and/or C-terminal protecting groups with similar properties to acetyl or amide groups.

Derivatives and analogues of peptides according to the invention may also include those that increase the peptide's half-life in vivo. For example, a derivative or analogue of the peptides of the invention may include peptoid and retropeptoid derivatives of the peptides, peptide-peptoid hybrids and D-amino acid derivatives of the peptides.

Peptoids, or poly-N-substituted glycines, are a class of peptidomimetics whose side chains are appended to the nitrogen atom of the peptide backbone, rather than to the alpha-carbon, as they are in amino acids. Peptoid derivatives of the peptides of the invention may be readily designed from knowledge of the structure of the peptide. Retropeptoids (in which all amino acids are replaced by peptoid residues in reversed order) are also suitable derivatives in accordance with the invention. A retropeptoid is expected to bind in the opposite direction in the ligand-binding groove, as compared to a peptide or peptoid-peptide hybrid containing one peptoid residue. As a result, the side chains of the peptoid residues are able point in the same direction as the side chains in the original peptide.

The term “derived from” can mean an amino acid sequence, which is a derivative or a modification of an amino acid sequence that is present in, or forms, the C-terminus of AChE, and portion thereof.

The term “truncation thereof” can mean the cyclic polypeptide derived from AChE is reduced in size by the removal of amino acids. The reduction of amino acids may be achieved by removal of residues from the C- or N-terminal of the peptide prior to cyclisation into the cyclic polypeptide of the invention, or may be achieved by deletion of one or more amino acids from within the core of the peptide prior to cyclisation.

Acetylcholinesterase is a serine protease that hydrolyses acetylcholine, and will be well-known to the skilled person. The major form of acetylcholinesterase, which is found in the brain, is known as tailed acetylcholinesterase (T-AChE). The protein sequence of one embodiment of human tailed acetylcholinesterase (Gen Bank: AAA68151.1) is 614 amino acids in length, and is provided herein as SEQ ID No:1, as follows:

| [SEQ ID No: 1] | |
|---|---|
| 1 | mrppqcllht pslaspllll llwllgggvg aegredaell vtvrggrlrg irlktpggpv |
| 61 | saflgipfae ppmgprrflp pepkqpwsgv vdattfqsvc yqyvdtlypg fegtemwnpn |
| 121 | relsedclyl nvwtpyprpt sptpvlvwiy gggfysgass ldvydgrflv qaertvlvsm |
| 181 | nyrvgafgfl alpgsreapg nvglldqrla lqwvqenvaa fggdptsvtl fgesagaasv |
| 241 | gmhllsppsr glfhravlqs gapngpwatv gmgearrrat qlahlvgcpp ggtggndtel |
| 301 | vaclrtrpaq vlvnhewhvl pqesvfrfsf vpvvdgdfls dtpealinag dfhglqvlvg |
| 361 | vvkdegsyfl vygapgfskd neslisraef lagvrvgvpq vsdlaaeavv lhytdwlhpe |
| 421 | dparlreals dvvgdhnvvc pvaqlagrla aqgarvyayv fehrastlsw plwmgvphgy |
| 481 | eiefifgipl dpsrnytaee kifaqrlmry wanfartgdp neprdpkapq wppytagaqq |
| 541 | yvsldlrple vrrglraqac afwnrflpkl lsatdtldea erqwkaefhr wssymvhwkn |
| 601 | qfdhyskqdr csdl |

It will be appreciated that the first 31 amino acid residues of SEQ ID No:1 are removed while the protein is released, thereby leaving a 583 amino acid sequence. Accordingly, it is preferred that the cyclic polypeptide, derivative or analogue thereof comprises or consists of an amino acid sequence derived from the C-terminus of acetylcholinesterase, or a truncation thereof, wherein the acetylcholinesterase comprises an amino acid sequence substantially as set out in SEQ ID No:1, preferably excluding the 31 amino acids at the N-terminal.

Preferably, the cyclic polypeptide, derivative or analogue thereof comprises or consists of an amino acid sequence derived from the last 300, 200, 100 or 50 amino acids forming the C-terminus of acetylcholinesterase, or a truncation thereof, most preferably wherein the acetylcholinesterase comprises or consists of an amino acid sequence substantially as set out in SEQ ID No:1. The cyclic polypeptide, derivative or analogue thereof preferably comprises or consists of an amino acid sequence derived from the last 40 amino acids forming the C-terminus of acetylcholinesterase, or a truncation thereof. The cyclic polypeptide, derivative or analogue thereof preferably comprises or consists of an amino acid sequence derived from the last 30 amino acids forming the C-terminus of acetylcholinesterase, or a truncation thereof.

The cyclic polypeptide, derivative or analogue thereof may comprise or consist of between 4 and 50 amino acids, preferably between 8 and 40 amino acid residues, preferably between 10 and 30 amino acids, more preferably between 9 and 20 amino acids, and most preferably between 10 and 16 amino acids. More preferably, the cyclic polypeptide, derivative or analogue thereof may comprise or consist of between 13 and 15 amino acid residues.

Preferably, the cyclic polypeptide, derivative or analogue thereof, comprises between 4 and 50 amino acid residues, between 4 and 40 amino acid residues, between 4 and 35 amino acid residues, between 4 and 32 amino acid residues, between 4 and 30 amino acid residues, between 4 and 25 amino acid residues, between 4 and 20 amino acid residues, or between 4 and 15 amino acid residues.

Preferably, the cyclic polypeptide, derivative or analogue thereof, comprises between 5 and 50 amino acid residues, between 5 and 40 amino acid residues, between 5 and 35 amino acid residues, between 5 and 32 amino acid residues, between 5 and 30 amino acid residues, between 5 and 25 amino acid residues, between 5 and 20 amino acid residues, or between 5 and 15 amino acid residues.

Preferably, the cyclic polypeptide, derivative or analogue thereof, comprises between 6 and 50 amino acid residues, between 6 and 40 amino acid residues, between 6 and 35 amino acid residues, between 6 and 32 amino acid residues, between 6 and 30 amino acid residues, between 6 and 25 amino acid residues, between 6 and 20 amino acid residues, or between 6 and 15 amino acid residues.

Preferably, the cyclic polypeptide, derivative or analogue thereof, comprises between 8 and 50 amino acid residues, between 8 and 40 amino acid residues, between 8 and 35 amino acid residues, between 8 and 30 amino acid residues, between 8 and 30 amino acid residues, between 8 and 25 amino acid residues, between 8 and 20 amino acid residues, or between 8 and 15 amino acid residues.

Preferably, the cyclic polypeptide, derivative or analogue thereof, comprises between 9 and 50 amino acid residues, between 9 and 40 amino acid residues, between 9 and 35 amino acid residues, between 9 and 30 amino acid residues, between 9 and 25 amino acid residues, between 9 and 20 amino acid residues, or between 9 and 15 amino acid residues.

Preferably, the cyclic polypeptide, derivative or analogue thereof, comprises between 10 and 50 amino acid residues, between 10 and 40 amino acid residues, between 10 and 35 amino acid residues, between 10 and 30 amino acid residues, between 10 and 25 amino acid residues, between 10 and 20 amino acid residues, or between 10 and 15 amino acid residues.

Preferably, the cyclic polypeptide, derivative or analogue thereof, comprises between 11 and 50 amino acid residues, between 11 and 40 amino acid residues, between 11 and 35 amino acid residues, between 11 and 30 amino acid residues, between 11 and 25 amino acid residues, between 11 and 20 amino acid residues, or between 11 and 15 amino acid residues.

Preferably, the cyclic polypeptide, derivative or analogue thereof, comprises between 12 and 50 amino acid residues, between 12 and 40 amino acid residues, between 12 and 35 amino acid residues, between 12 and 30 amino acid residues, between 12 and 25 amino acid residues, between 12 and 20 amino acid residues, or between 12 and 15 amino acid residues.

Preferably, the cyclic polypeptide, derivative or analogue thereof, comprises between 13 and 50 amino acid residues, between 13 and 40 amino acid residues, between 13 and 35 amino acid residues, between 13 and 30 amino acid residues, between 13 and 25 amino acid residues, between 13 and 20 amino acid residues, or between 13 and 15 amino acid residues.

Preferably, the cyclic polypeptide, derivative or analogue thereof, comprises between 14 and 50 amino acid residues, between 14 and 40 amino acid residues, between 14 and 35 amino acid residues, between 14 and 30 amino acid residues, between 14 and 25 amino acid residues, between 14 and 20 amino acid residues, or between 14 and 15 amino acid residues.

The inventor has prepared three peptide sequences that are derived from the C-terminus of AChE, and which are referred to herein as T30, T14 and T15, where the number corresponds to the amino acid number.

The amino acid sequence of T30 (which corresponds to the last 30 amino acid residues of SEQ ID No:1) is provided herein as SEQ ID No:2, as follows:—

[SEQ ID No: 2]
KAEFHRWSSYMVHWKNQFDHYSKQDRCSDL

The amino acid sequence of T14 (which corresponds to the 14 amino acid residues located towards the end of SEQ ID No:1, and lacks the final 15 amino acids found in T30) is provided herein as SEQ ID No:3, as follows:—

[SEQ ID No: 3]
AEFHRWSSYMVHWK

The amino acid sequence of T15 (which corresponds to the last 15 amino acid residues of SEQ ID No:1) is provided herein as SEQ ID No:4, as follows:—

[SEQ ID No: 4]
NQFDHYSKQDRCSDL

It will be appreciated that any of the polypeptides or peptides described herein may be synthesised de novo using standard peptide synthesis methods commonly known to the skilled person, and, as such, may then be used in any of the cosmetic/therapeutic applications described herein. Accordingly, any of the peptides can be produced by forming a peptide bond between adjacent amino acids of the sequences provided herein to build up to the full sequence length, i.e. the first amino acid is provided, to which a second amino acid is attached, and so on up to the desired length of peptide. Therefore, it is not necessary to start with the full acetyl cholinesterase sequence, or a shorter truncation thereof, and reduce the length of the polypeptide by removing amino acids from the N- and/or C-terminal until the desired peptide length is reached. Indeed, for the sake of speed, convenience and cost, it is preferred that the polypeptide is created using a de novo peptide synthesis method.

It will be appreciated that any of the sequences represented as SEQ ID No:2-4 can be readily cyclised (or cyclated) to form a cyclic polypeptide of the first aspect. For example, cyclization of peptides can be achieved by side-chain-to-side-chain, side-chain-to-backbone, or head-to-tail (C-terminus to N-terminus) cyclization techniques. In one preferred embodiment, head-to-tail cyclization is the preferred method by which the cyclic polypeptides are produced. The cyclic polypeptides may be synthesised using either classical solution-phase linear peptide cyclization or resin-based cyclization. Preferred methods for cyclization are described in the Examples. In another preferred embodiment, the polypeptide is produced using a cyclization cleavage approach, in which the cyclic polypeptide is synthesized by cyclization after step-wise linear peptide synthesis. An advantage of this method is that the side-chain does not need to be anchored, making the approach more general. Preferably, prior to use, resultant samples of cyclic peptides can be analysed by MALDI-TOF MS.

Accordingly, a preferred polypeptide according to the invention comprises or consists of cyclic SEQ ID No:2, 3 or 4, or a functional variant or fragment thereof.

The inventors found that cyclized SEQ ID No: 3 (i.e. referred to herein as “cyclized T14”, “CT14” or “NBP-14”) surprisingly inhibits T30-induced intracellular calcium influx in keratinocytes.

Accordingly, a most preferred cyclic polypeptide used in the invention described herein comprises or consists of cyclic SEQ ID No:3, or a functional variant or fragment thereof.

It will be appreciated that the cyclic polypeptide according to the invention may be used in a medicament, which may be used as a monotherapy (i.e. use of the cyclic polypeptide, derivative or analogue thereof alone), for treating, ameliorating, or preventing a skin condition. Alternatively, the cyclic polypeptide derivative or analogue thereof according to the invention may be used as an adjunct to, or in combination with, known therapies for treating, ameliorating, or preventing a skin condition.

The cyclic polypeptide derivative or analogue thereof according to the invention may be combined in compositions having a number of different forms depending, in particular, on the manner in which the composition is to be used. Thus, for example, the composition may be in the form of a powder, tablet, capsule, liquid, ointment, cream, gel, hydrogel, aerosol, spray, micellar solution, transdermal patch, liposome suspension or any other suitable form that may be administered to a person or animal in need of treatment. It will be appreciated that the vehicle of medicaments according to the invention should be one which is well-tolerated by the subject to whom it is given, and preferably enables delivery of the cyclic polypeptide to the skin.

Cyclic polypeptides according to the invention may also be incorporated within a slow- or delayed-release device. Such devices may, for example, be inserted on or under the skin, and the medicament may be released over weeks or even months. The device may be located at least adjacent the treatment site. Such devices may be particularly advantageous when long-term treatment with cyclic polypeptides used according to the invention is required and which would normally require frequent administration (e.g. at least daily injection).

In a preferred embodiment, medicaments according to the invention may be administered topically to the skin, preferably directly at a site requiring treatment.

It will be appreciated that the amount of the cyclic polypeptide that is required is determined by its biological activity and bioavailability, which in turn depends on the mode of administration, the physiochemical properties of the cyclic polypeptide and whether it is being used as a monotherapy or in a combined therapy. The frequency of administration will also be influenced by the half-life of the cyclic polypeptide within or on the subject being treated. Optimal dosages to be administered may be determined by those skilled in the art, and will vary with the particular cyclic polypeptide in use, the strength of the pharmaceutical composition, and the mode of administration. Additional factors depending on the particular subject being treated will result in a need to adjust dosages, including subject age, weight, gender, diet, and time of administration.

Generally, a daily dose of between 0.001 μg/kg of body weight and 10 mg/kg of body weight, or between 0.01 μg/kg of body weight and 1 mg/kg of body weight, of the cyclic polypeptide according to the invention may be used for treating, ameliorating, or preventing a skin condition, depending upon which cyclic polypeptide is used.

The cyclic polypeptide may be administered before, during or after onset the skin condition. Daily doses may be given as a single administration (e.g. a single daily application). Alternatively, the cyclic polypeptide may require administration twice or more times during a day. As an example, cyclic polypeptides may be administered as two (or more depending upon the severity of the skin condition being treated) daily doses of between 0.07 μg and 700 mg (i.e. assuming a body weight of 70 kg). A patient receiving treatment may take a first dose upon waking and then a second dose in the evening (if on a two dose regime) or at 3- or 4-hourly intervals thereafter. Alternatively, a slow release device may be used to provide optimal doses of cyclic polypeptide according to the invention to a patient without the need to administer repeated doses.

Known procedures, such as those conventionally employed by the pharmaceutical industry (e.g. in vivo experimentation, clinical trials, etc.), may be used to form specific formulations of the cyclic polypeptide according to the invention and precise therapeutic regimes (such as daily doses of the agents and the frequency of administration). The inventors believe that they are the first to suggest a skin condition treatment composition, based on the use of a cyclic polypeptide of the invention.

Hence, in a fourth aspect of the invention, there is provided a skin condition treatment pharmaceutical composition comprising a therapeutically effective amount of the cyclic polypeptide, derivative or analogue thereof comprising an amino acid sequence derived from the C-terminus of acetylcholinesterase (AChE), or a truncation thereof, and a pharmaceutically acceptable vehicle.

The invention also provides in a fifth aspect, a process for making the skin condition treatment composition according to the fourth aspect, the process comprising combining a therapeutically effective amount of the cyclic polypeptide, derivative or analogue thereof comprising an amino acid sequence derived from the C-terminus of acetylcholinesterase (AChE), or a truncation thereof, with a pharmaceutically acceptable vehicle.

The cyclic polypeptide, derivative or analogue thereof preferably comprises or consists of Cyclic T14 (i.e. NBP-14) as disclosed herein, i.e. SEQ ID No: 3.

The inventors' discovery that cyclic peptides derived from the C-terminus of acetylcholinesterase may reduce keratinocyte proliferation, indicates that the cyclic peptide may also be advantageously be used to reduce scar formation and therefore be used for cosmetic purposes. In some embodiments, the anti-scarring activity may be therapeutic.

Thus, in a sixth aspect of the invention there is provided a cyclic polypeptide, derivative or analogue thereof comprising an amino acid sequence derived from the C-terminus of acetylcholinesterase (AChE), or a truncation thereof, for use in reducing, preventing or inhibiting scar formation.

In a seventh aspect there is provided a method of reducing, inhibiting or preventing scar formation, the method comprising, administering, or having administered to a subject in need of such treatment, a therapeutically effective amount of a cyclic polypeptide, derivative or analogue thereof comprising an amino acid sequence derived from the C-terminus of acetylcholinesterase (AChE), or a truncation thereof.

Preferably, the cyclic polypeptide, derivative or analogue thereof is as defined in the first aspect.

However, in other embodiments, the anti-scarring activity may be cosmetic.

Accordingly, in an eighth aspect of the invention, there is provided a method for cosmetic treatment of skin, comprising applying, to skin, a cyclic polypeptide, or a derivative or analogue thereof comprising an amino acid sequence derived from the C-terminus of acetylcholinesterase (AChE), or a truncation thereof.

Preferably, the cyclic polypeptide, derivative or analogue thereof is as defined in the first aspect.

Cosmetic treatment of skin may include reducing, preventing or inhibiting scar formation.

In an ninth aspect of the invention, there is provided the use of a cyclic polypeptide, or a derivative or analogue thereof comprising an amino acid sequence derived from the C-terminus of acetylcholinesterase (AChE), or a truncation thereof, for cosmetic treatment of skin.

The invention also extends to cosmetic compositions.

Hence, in a tenth aspect of the invention, there is provided a cosmetic composition comprising a cosmetically effective amount of the cyclic polypeptide, derivative or analogue thereof comprising an amino acid sequence derived from the C-terminus of acetylcholinesterase (AChE), or a truncation thereof, and a cosmetically acceptable vehicle.

The invention also provides in an eleventh aspect, a process for making the cosmetic composition according to the tenth aspect, the process comprising combining a cosmetically effective amount of the cyclic polypeptide, derivative or analogue thereof comprising an amino acid sequence derived from the C-terminus of acetylcholinesterase (AChE), or a truncation thereof, with a cosmetically acceptable vehicle.

### Prevention, Reduction or Inhibition of Scarring

The prevention, reduction or inhibition of scarring within the context of the present invention should be understood to encompass any degree of prevention, reduction or inhibition in scarring achieved on healing of a treated wound, as compared to the level of scarring occurring on healing of a control-treated or untreated wound. Throughout the specification references to “prevention”, “reduction” or “inhibition” of scarring are generally to be taken, except where the context requires otherwise, to represent substantially equivalent activities, involving equivalent mechanisms mediated by polypeptides of the present invention.

### Assessment of Scarring

The extent of inhibition of scarring that may be required in order to achieve a therapeutic effect will be apparent to, and may readily be determined by, a clinician responsible for the care of the patient. The clinician may undertake a suitable determination of the extent of inhibition of scarring that has been achieved, in order to assess whether or not a therapeutic effect has been achieved, or is being achieved. Such an assessment may, but need not necessarily, be made with reference to suggested methods of measurement described herein.

The extent to which inhibition of scarring after wound closure is achieved may be assessed with reference to the effects that such an active agent may achieve in human patients treated with the methods or medicaments of the invention. Alternatively, inhibition of scarring that may be achieved may be assessed with reference to experimental investigations using suitable in vitro or in vivo models. The use of experimental models to investigate inhibition of scarring may be particularly preferred in assessing the therapeutic effectiveness of the cyclic peptides of the present invention, or in establishing therapeutically effective amounts of such polypeptides.

Animal models of wound healing and scar formation represent preferred experimental models for in vivo assessment of the extent of scar inhibition that may be achieved using the medicaments or methods of the invention. Examples of such models are described below for illustrative purposes. The models of scarring and methods for assessing scarring described herein may be used to determine therapeutically effective polypeptides.

Inhibition of scarring, using the cyclic peptides of the invention, can be effected at any body site and in any tissue or organ so far investigated. For illustrative purposes the scar inhibitory activity of cyclic peptides and methods of the invention will primarily be described with reference to inhibition of scarring that may be brought about in the skin (the body's largest organ). However, the skilled person will immediately appreciate that many of the factors that are relevant when considering inhibition of scarring in the skin are also relevant to inhibition of scarring in other organs or tissues. Accordingly the skilled person will recognise that, except for where the context requires otherwise, the parameters and assessments considered below in respect of scars of the skin may also be applicable to scarring in tissues other than the skin. The skilled person will recognise that the above is equally applicable in the context of re-epithelisation and the rate of wound healing and is not limited to the assessment of scarring.

In the skin, treatment of wounds may improve the macroscopic and microscopic appearance of scars which arise when these wounds close; macroscopically the scars may be less visible and blend with the surrounding skin, microscopically the collagen fibres within the scar may have morphology and anisotropic organisation that is more similar to those in the surrounding skin.

The inhibition of scarring achieved using methods and cyclic peptides of the invention may be assessed and/or measured with reference to either the microscopic or macroscopic appearance of a scar generated by treatment of a wound to promote closure as compared to the appearance of a scar formed by closure of a wound with no polypeptide treatment. Inhibition of scarring may also suitably be assessed with reference to both macroscopic and microscopic appearance of a treated scar.

In considering the macroscopic appearance of a scar resulting from a treated wound, the extent of scarring, and hence the magnitude of any inhibition of scarring achieved, may be assessed with reference to any of a number of parameters. Most preferably, holistic assessment of the scar by means of assessment of macroscopic photographs by an independent expert panel, by means of an independent lay panel or clinically by means of a macroscopic assessment by a clinician of the patients themselves. Assessments are captured by means of a VAS (visual analogue scale) or a categorical scale.

Macroscopic characteristics of a scar which can be assessed objectively include: i) Colour of the scar. Scars may typically be hypopigmented or hyperpigmented with regard to the surrounding skin. Inhibition of scarring may be demonstrated when the pigmentation of a treated scar more closely approximates that of unscarred skin than does the pigmentation of an untreated scar. Similarly, scars may be redder than the surrounding skin. In this case inhibition of scarring may be demonstrated when the redness of a treated scar fades earlier, or more completely, or to resemble more closely the appearance of the surrounding skin, compared to an untreated scar. There are a number of non-invasive colorimetric devices which are able to provide data with respect to pigmentation of scars and unscarred skin, as well as redness of the skin (which may be an indicator of the degree of vascularity present in the scar or skin). Examples of such devices include the X-rite SP-62 spectrophotometer, Minolta Chronometer CR-200/300; Labscan 600; Dr. Lange Micro Colour; Derma Spectrometer; laser-Doppler flow meter; and Spectrophotometric intracutaneous Analysis (SIA) scope. ii) Height of the scar. Scars may typically be either raised or depressed as compared to the surrounding skin. Inhibition of scarring may be demonstrated when the height of a treated scar more closely approximates that of unscarred skin (i.e. is neither raised nor depressed) than does the height of an untreated scar. Height of the scar can be measured directly on a patient by means of profilometry, or indirectly, by profilometry of moulds taken from a scar. iii) Surface texture of the scar. Scars may have surfaces that are relatively smoother than the surrounding skin (giving rise to a scar with a “shiny” appearance) or that are rougher than the surrounding skin. Inhibition of scarring may be demonstrated when the surface texture of a treated scar more closely approximates that of unscarred skin than does the surface texture of an untreated scar. Surface texture can be measured directly on a patient by means of profilometry, or indirectly by profilometry of moulds taken from a scar. iv) Stiffness of the scar. The abnormal composition and structure of scars means that they are normally stiffer than the undamaged skin surrounding the scar. In this case, inhibition of scarring may be demonstrated when the stiffness of a treated scar more closely approximates that of unscarred skin than does the stiffness of an untreated scar.

A treated scar will preferably exhibit inhibition of scarring as assessed with reference to at least one of the parameters for macroscopic assessment set out in the present specification. More preferably a treated scar may demonstrate inhibited scarring with reference to at least two parameters, even more preferably at least three parameters, and most preferably at least four of these parameters (for example, all four of the parameters set out above). The parameters described above may be used in the development of a visual analogue scale (VAS) for the macroscopic assessment of scarring. Details regarding implementation of VASs are described below. Microscopic assessment may also provide a suitable means by which the quality of treated and untreated or control scars may be compared. Microscopic assessment of scar quality may typically be carried out using histological sections of scars. Suitable parameters for the microscopic assessment of scars may include: i) Thickness of extracellular matrix (ECM) fibres. Inhibition of scarring may be demonstrated when the thickness of ECM fibres in a treated scar more closely approximates the thickness of ECM fibres found in unscarred skin than does the thickness of fibres found in an untreated scar. ii) Orientation of ECM fibres. ECM fibres found in scars tend to exhibit a greater degree of alignment with one another than do those found in unscarred skin (which have a random orientation frequently referred to as “basket weave”). Accordingly, inhibition of scarring may be demonstrated when the orientation of ECM fibres in a treated scar more closely approximates the orientation of ECM fibres found in unscarred skin than does the orientation of such fibres found in an untreated scar. iii) ECM composition of the scar. The composition of ECM molecules present in scars shows differences from that found in normal skin, with a reduction in the amount of elastin present in ECM of scars. Thus inhibition of scarring may be demonstrated when the composition of ECM fibres in the dermis of a treated scar more closely approximates the composition of such fibres found in unscarred skin than does the composition found in an untreated scar. iv) Cellularity of the scar. Scars tend to contain relatively fewer cells than does unscarred skin. It will therefore be appreciated that inhibition of scarring may be demonstrated when the cellularity of a treated scar more closely approximates the cellularity of unscarred skin than does the cellularity of an untreated scar. v) Appendages. Scars do not contain adnexal structures such as glands or hair follicles. The presence of these in the treated skin will indicate that functional tissue regeneration rather than scar formation has occurred.

Other features that may be taken into account in assessing the microscopic quality of scars include elevation or depression of the scar relative to the surrounding unscarred skin, and the prominence or visibility of the scar at the interface with the unscarred skin.

The parameters described above may be used in generating a VAS for the microscopic assessment of scarring. Such a VAS may consider collagen organisation and abundance in the papillary dermis and the reticular dermis may also provide a useful index of scar quality. Inhibition of scarring may be indicated when the quality of a treated scar is closer to that of unscarred skin than is the quality of an untreated or control scar. It is surprising to note that the overall appearance of scars, such as those of the skin, is little influenced by the epidermal covering of the scar, even though this is the part of the scar that is seen by the observer. Instead, the inventors find that the properties of the connective tissue (such as that making up the dermis, or neo-dermis) present within the scar have greater impact on the perception of extent of scarring, as well as on the function of the scarred tissue. Accordingly assessments of criteria associated with the connective tissues such as the dermis, rather than epidermis, may prove to be the most useful in determining inhibition of scarring.

The thickness of ECM fibres and orientation of ECM fibres may be favoured parameters, for assessing inhibition of scarring. A treated scar may preferably have improved ECM orientation (i.e. orientation that is more similar to unscarred skin than is the orientation in an untreated scar).

A treated scar will preferably demonstrate inhibition of scarring as assessed with reference to at least one of the parameters for microscopic assessment set out above. More preferably a treated scar may demonstrate inhibition of scarring with reference to at least two of the parameters, even more preferably at least three of the parameters, even more preferably at least four of the parameters, and most preferably all five of these parameters.

It will be appreciated that inhibition of scarring achieved using the cyclic peptides or methods of the invention may be indicated by improvement of one or more suitable parameters combined from different assessment schemes (e.g. inhibition as assessed with reference to at least one parameter used in macroscopic assessment and at least one parameter used in microscopic assessment).

Further examples of suitable parameters for the clinical measurement and assessment of scars may be selected based upon a variety of measures or assessments including those described by Duncan et al. (2006), Beausang et al. (1998) and van Zuijlen et al (2002). Except for where the context requires otherwise, many of the following parameters may be applied to macroscopic and/or microscopic assessment of scarring. Examples of Suitable parameters for assessment of scars in the skin may include:

1. Assessment with Regard to Visual Analogue Scale (VAS) Scar Score.

Prevention, reduction or inhibition of scarring may be demonstrated by a reduction in the VAS score of a treated scar when compared to a control scar. A suitable VAS for use in the assessment of scars may be based upon the method described by Duncan et al. (2006) or by Beausang et al. (1998). This is typically a 10 cm line in which 0 cm is considered an imperceptible scar and 10 cm a very poor hypertrophic scar.

2. Assessment with Regard to a Categorical Scale.

Prevention, reduction or inhibition of scarring may be determined by allocating scars to different categories based on either textual descriptions e.g. “barely noticeable”, “blends well with normal skin”, “distinct from normal skin”, etc., by comparing a treated scar and a an untreated or control scar, noting any differences between these, and allocating the differences to selected categories (suitable examples of which may be “mild difference”, “moderate difference”, “major difference”, etc.). Assessment of this sort may be performed by the patient, by an investigator, by an independent panel, or by a clinician, and may be performed either directly on the patient or on photographs or moulds taken from the patient. Inhibition of scarring may be demonstrated when an assessment indicates that treated scars are generally allocated to more favourable categories than are untreated or control scars.

### 3. Scar Height, Scar Width, Scar Perimeter, Scar Area or Scar Volume.

The height and width of scars can be measured directly upon the subject, for example by use of manual measuring devices such as callipers, or automatically with the use of profilometers. Scar width, perimeter and area may be measured either directly on the subject, by image analysis of photographs of the scar, by analysis of silicone mould impressions of the scar, or by analysis of positive casts made from such impressions. The skilled person will also be aware of further non-invasive methods and devices that can be used to investigate suitable parameters, including silicone moulding, ultrasound, optical three-dimensional profilimetry and high resolution Magnetic Resonance Imaging. Inhibition of scarring may be demonstrated by a reduction in the height, width, area, perimeter or volume, or any combination thereof, of a treated scar as compared to an untreated scar.

### 4. Scar Distortion and Mechanical Performance

Scar distortion may be assessed by visual comparison of a scar and unscarred skin. A suitable comparison may categorise a selected scar as causing no distortion, mild distortion, moderate distortion or severe distortion.

The mechanical performance of scars can be assessed using a number of non-invasive methods and devices based upon suction, pressure, torsion, tension and acoustics. Suitable examples of devices capable of use in assessing mechanical performance of scars include Indentometer, Cutometer, Reviscometer, Visco-elastic skin analysis, Dermaflex, Durometer, Dermal Torque Meter and Elastometer.

Inhibition of scarring may be demonstrated by a reduction in distortion caused by treated scars as compared to that caused by untreated scars. It will also be appreciated that inhibition of scarring may be demonstrated by the mechanical performance of unscarred skin being more similar to that of treated scars than of untreated scars.

### Photographic Assessments Independent Lay Panel

Photographic assessment of treated and untreated scars may be performed by an independent lay panel of assessors using standardised and calibrated photographs of the scars. The scars may be assessed by an independent lay panel to provide categorical ranking data (e.g. that a given treated scar is “better”, “worse” or “no different” when compared to an untreated scar) and quantitative data using a Visual Analogue Scale (VAS) based upon the method described by Duncan et al. (2006) and Beausang et al. (1998).

### Expert Panel

Photographic assessment of treated and untreated scars may alternatively or additionally be performed by a panel of expert assessors using standardised and calibrated photographs of the scars to be assessed, and/or positive casts of silicone moulds. The panel of experts may preferably consist of individuals skilled in the art, suitable examples of which include plastic surgeons, dermatologists or scientists having relevant technical backgrounds.

### Clinical Assessment

A clinician, or an independent panel of clinicians may assess the scar(s) on a patient using any of the forgoing parameters e.g. VAS, colour, categorical scales, etc. A suitable clinician may be a clinician responsible for care of a patient, or may be a clinician investigating efficacy of therapies for inhibition of scarring.

### Patient Assessment

A patient may assess their own scars and/or compare scars by means of a structured questionnaire. A suitable questionnaire may measure parameters such as: the patient's satisfaction with their scar; how well the scar blends with the unscarred skin; as well as the effect of the scar on their daily life (suitable questions may consider whether the patient uses clothes to hide the scar, or otherwise avoids exposing it) and/or scar symptoms (examples of which may include itch, pain or paresthesia). Inhibition of scarring may be indicated by the treated scar receiving a more positive rating from the patient, and/or causing the patient fewer problems, and/or causing fewer or less scar symptoms, and/or an increase in patient satisfaction compared to an untreated scar. In addition to categorical data, quantitative data (preferably relating to the above parameters) can be generated using image analysis in combination with suitable visualisation techniques. Examples of suitable visualisation techniques that may be employed in assessing scar quality are specific histological stains or immuno-labelling, wherein the degree of staining or labelling present may be quantitatively determined by image analysis.

Quantitative data may be usefully and readily produced in relation to the following parameters:
1. Scar width, height, elevation, volume and area.
2. Collagen organisation, collagen fibre thickness, collagen fibre density.
3. Number and orientation of fibroblasts.
4. Quantity and orientation of other ECM molecules e.g. elastin, fibronectin

Prevention, reduction or inhibition of scarring may be demonstrated by a change in any of the parameters considered above such that a treated scar more closely resembles unscarred skin than does a control or untreated scar (or other suitable comparator). The assessments and parameters discussed above are suitable for assessment of the effects of a polypeptide, on scar formation, as compared to control, placebo or standard care treatment in animals or humans. It will be appreciated that these assessments and parameters may be utilised in determining a therapeutically effective polypeptide that may be used for scar prevention, reduction or inhibition; and in determining therapeutically effective amounts of polypeptides of the invention, such as AXL. Appropriate statistical tests may be used to analyse data sets generated from different treatments in order to investigate the significance of results.

Other parameters that may be used in the assessment of scarring in organs other than the skin may be determined with reference to the organ in question. For example, corneal scarring may be assessed by measuring the opacity, or transmitting/refractory properties, of the cornea and measurement of corneal curvature. Such assessments may, for example, be made using in vivo confocal microscopy and/or specular microscopy or corneal topography.

Successful inhibition of scarring in tendons or ligaments may be indicated by restoration of function of tissues treated with the medicaments or methods of the invention. Suitable indicators of function may include the ability of the tendon or ligament to bear weight, stretch, flex, etc. Such assessments may, for example, be made using electrophysiological reflex examination, surface electromyography, ultrasonography, ultrasound/MRI scan, and self-reported symptom and pain questionnaires.

The extent of scarring occurring in blood vessels can be measured directly e.g. using ultrasound, or indirectly by means of blood flow. Inhibition of scarring achieved using the medicaments or methods of the invention may lead to a reduction in narrowing of the blood vessel lumen and allow a more normal blood flow.

### Wound Sites

The wound may be present at any body site, and in any tissue or organ, where a wound may occur. The skin represents the preferred site at which scar formation is prevented, reduced or inhibited. The inventors believe that the cyclic peptides of the present invention may beneficially reduce scar formation in all types of epithelial wounds. Examples of specific wounds in which the effects of the invention may be seen include wounds selected from the group consisting of wounds of the skin (such as burns, incision wounds, pressure ulcers), the lungs, the eye (including the inhibition of scarring resulting from eye surgery such as LASIK surgery, LASEK surgery, PRK surgery, glaucoma filtration surgery, cataract surgery, or surgery in which the lens capsule may be subject to scarring) such as those giving rise to corneal cicatrisation; wounds subject to capsular contraction (which is common surrounding breast implants); wounds of the oral cavity, including the lips and palate (for example, to inhibit scarring resulting from treatment of cleft lip or palate or to promote closure or oral ulcers); wounds of the internal organs such as the digestive tissues and reproductive tissues; wounds of body cavities such as the abdominal cavity, pelvic cavity and thoracic cavity (where inhibition of scarring may reduce the number of incidences of adhesion formation and/or the size of adhesions formed); and surgical wounds (in particular wounds associated with cosmetic procedures, such as scar revision or isolation of strip grafts for hair transplant surgery). It is particularly preferred that the cyclic peptides of the present invention be used to prevent, reduce or inhibit scarring associated with wounds of the skin.

Incisional wounds are a preferred group of wounds resulting in scarring which may be inhibited by the polypeptides of the invention. Surgical incisional wounds may constitute a particularly preferred group of wounds in respect of which scarring may be inhibited utilising the medicaments and methods of the invention.

Cyclic peptides of the present invention may be used to inhibit scarring associated with plastic or cosmetic surgery. Since a large number of plastic or cosmetic surgeries consist of elective surgical procedures it is readily possible to administer a polypeptide of the present invention, prior to surgery, and/or around the time of closure of the wound (for instance, before or after the application of sutures), and this use represents a particularly preferred embodiment of the invention.

In surgical procedures in general, a preferred route by which a cyclic peptide of the present invention may be administered is via localised injection (such as intradermal injection). Such injections may form raised blebs, which may then be incised as part of the surgical procedure, or alternatively the bleb may be raised by injecting the wound margins after the wound has been closed e.g. by sutures. Alternatively, the cyclic peptide may be administered in a cream formulation or in a bandage, or may be coated on the sutures used for incision closure.

Scar revisions are surgical procedures in which existing scars are “revised” (for example through excision or realignment) in order to reduce the cosmetic and/or mechanical disruption caused by the existing scar. Probably the best known of these is “Z-plasty” in which two V-shaped flaps of skin are transposed to allow rotation of a line of tension. The use of the cyclic peptides of the invention in procedures associated with scar revision represents a preferred use in accordance with the present invention.

It is recognised that wounds resulting from burns injuries (which for the purposes of the present invention may be taken to encompass exposure to heated gasses or solids, as well as scalding injuries involving hot liquids; “freezer burn” injuries caused by exposure to extreme low temperatures; radiation burns; and chemical burns, such as those caused by caustic agents) may extend over great areas of an individual so afflicted. Accordingly, burns may give rise to scar formation covering a large proportion of a patient's body. This great extent of coverage increases the risk that the scar formed will cover areas of elevated cosmetic importance (such as the face, neck, arms or hands) or of mechanical importance (particularly the regions covering or surrounding joints). Burns injuries caused by hot liquids are frequently suffered by children (for example as a result of upsetting pans, kettles or the like) and, due to the relatively smaller body size of children, are particularly likely to cause extensive damage over a high proportion of the body area. Thus there is an elevated risk of both cosmetic and mechanical impairment associated with scarring after burns. After large burns, skin grafts are used as a treatment. This invention can be used in combination with a skin graft, to promote migration of epithelial cells from the graft to the uncovered wound, to quickly establish a barrier in non-grafted areas of skin.

A “subject” may be a vertebrate, mammal, or domestic animal. Hence, medicaments and cosmetics according to the invention may be used to treat any mammal, for example livestock (e.g. a horse), pets, or may be used in other veterinary applications. Most preferably, however, the subject is a human being.

A “therapeutically effective amount” of cyclic polypeptide is any amount which, when administered to a subject, is the amount of active agent that is needed to treat the skin condition, or produce the desired effect. The cyclic polypeptide, derivative or analogue thereof may be used as an adjuvant for the treatment of various skin conditions, such as eczema, psoriasis, melanoma, dermatitis and acne. This means that lower doses of other treatments would be required.

A “cosmetically effective amount” of cyclic polypeptide is any amount which, when administered to a subject, is the amount of active agent that is needed to produce the desired cosmetic effect.

For example, the therapeutically or cosmetically effective amount of cyclic polypeptide used may be from about 0.001 mg to about 800 mg, and preferably from about 0.01 mg to about 500 mg.

A “pharmaceutically acceptable vehicle” as referred to herein, is any known compound or combination of known compounds that are known to those skilled in the art to be useful in formulating pharmaceutical compositions.

A “cosmetically acceptable vehicle” as referred to herein, is any known compound or combination of known compounds that are known to those skilled in the art to be useful in formulating cosmetic compositions.

In one embodiment, the pharmaceutically acceptable vehicle or cosmetically acceptable vehicle may be a solid, and the composition may be in the form of a powder or tablet.

However, the pharmaceutical or cosmetic vehicle may be a liquid, and the pharmaceutical or cosmetic composition is in the form of a solution. Liquid pharmaceutical or cosmetic compositions, which are sterile solutions or suspensions, can be utilized by, for example, intramuscular, intrathecal, epidural, intraperitoneal, intravenous and particularly subcutaneous injection.

The cyclic polypeptide and compositions of the invention may be administered orally in the form of a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The cyclic polypeptide used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

Preferably, the cyclic polypeptide and compositions of the invention may be administered topically in the form of a cream, gel, lotion, ointment, cutaneous solution, suspension, spray, foam, bath additive, collodion, impregnated dressing or medicated plaster. The cream may be either oil-in-water or water-in-oil type. The cyclic polypeptide and compositions of the invention may be administered topically with emulgators such as alkyl sulphates, alkyl amines, alkyl pyrimidin compounds, etc. Acceptable oils for cream formulation include: white petrolatum, paraffin, cetearyl alcohol, cocoglycerides, cetyl alcohol, isopropyl miristate, cetyl palmitate, butyrum cacao, oleum helianthi, cera alba, lanolin, isopropyl palmitate, stearic acid, magnesium stearate. For preparation of a gel, the following gel forming additives may be used: cellulose gum (carboxymethyl cellulose), hydroxypropyl cellulose, methylcellulose, hydroxyethyl cellulose, ethylhydroxy cellulose, or laponite.

Preferably, the cyclic polypeptide and compositions of the invention may be administered topically with preserving agents, antioxidants, complexing agents, solvents, fragrances, bactericides, odor absorbers, vitamins, moisturizers, self-tanning compounds and anti-wrinkle active agents.

The compositions of the invention may contain cosmetically acceptable additives or adjuvants as well as cosmetic or dermatologic active agents. Representative additives and adjuvants include, for example, oil-soluble or oil-miscible solvents or co-solvents. Suitable examples of additives and adjuvants include, but are not limited to, fatty alcohols, fatty amides, alkylene carbonates, glycols, lower alcohols (e.g. ethanol, propanediol), dispersion enhancing agents, polymers, thickening agents, stabilizers, moisturizers, humectants, colorants, fillers, chelating agents, antioxidants (e.g. BHT, tocopherol), essential oils, fragrances, dyes, neutralizing or pH-adjusting agents (e.g., citric acid, triethylamine (TEA) and sodium hydroxide), preservatives, bactericides, conditioning or softening agents (e.g., panthenol and allantoin), extracts, such as botanical extracts, or any other ingredient commonly used in cosmetics for this type of application. Additives and adjuvants may be present in the compositions in amounts generally ranging from about 0.01% to about 10%, by weight. Examples of cosmetic active agents or dermatological active agents include free-radical scavengers, vitamins (e.g., Vitamin E and derivatives thereof), anti-elastase and anti-collagenase agents, peptides, fatty acid derivatives, steroids, trace elements, extracts of algae and of planktons, enzymes and coenzymes, flavonoids and ceramides, hydroxy acids and mixtures thereof, and enhancing agents. These ingredients may be soluble or dispersible in oil phase(s) that is/are present in the composition.

The cyclic polypeptide and compositions of the invention may be administered in a sterile solution or suspension containing other solutes or suspending agents (for example, enough saline or glucose to make the solution isotonic), bile salts, acacia, gelatin, sorbitan monoleate, polysorbate 80 (oleate esters of sorbitol and its anhydrides copolymerized with ethylene oxide) and the like. The cyclic polypeptide used according to the invention can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

It will be appreciated that the invention extends to any nucleic acid or peptide or variant, derivative or analogue thereof, which comprises substantially the amino acid or nucleic acid sequences of any of the sequences referred to herein, including functional variants or functional fragments thereof. The terms “substantially the amino acid/nucleotide/peptide sequence”, “functional variant” and “functional fragment”, can be a sequence that has at least 40% sequence identity with the amino acid/nucleotide/peptide sequences of any one of the sequences referred to herein, for example 40% identity with the sequence identified as SEQ ID No:1-4, and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence, which hybridizes to DNA sequences or their complements under stringent conditions. By stringent conditions, we mean the nucleotide hybridises to filter-bound DNA or RNA in 3× sodium chloride/sodium citrate (SSC) at approximately 45° ° C. followed by at least one wash in 0.2×SSC/0.1% SDS at approximately 20-65ºC. Alternatively, a substantially similar polypeptide may differ by at least 1, but less than 5, 10, 20, 50 or 100 amino acids from the sequences shown in SEQ ID No: 1-4.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:—

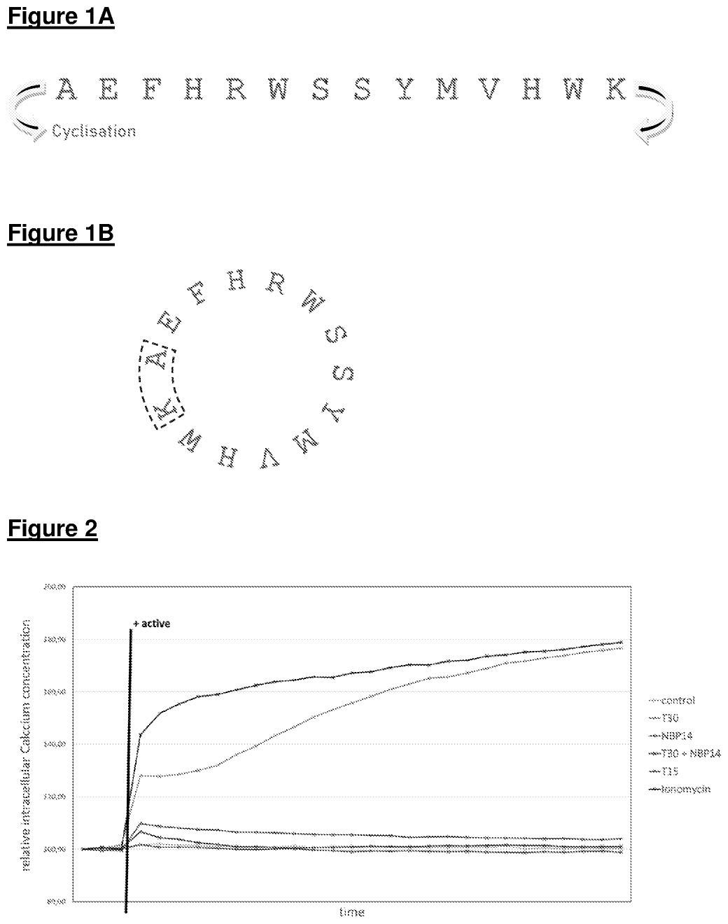

FIG. 1A shows the sequence of NBP-14 (SEQ ID No:3) with the terminal Alanine (A) and Lysine (K) residues forming the cyclisation sites. FIG. 1B shows the cyclic NBP-14 peptide in which the terminal Alanine and Lysine residues are linked together;

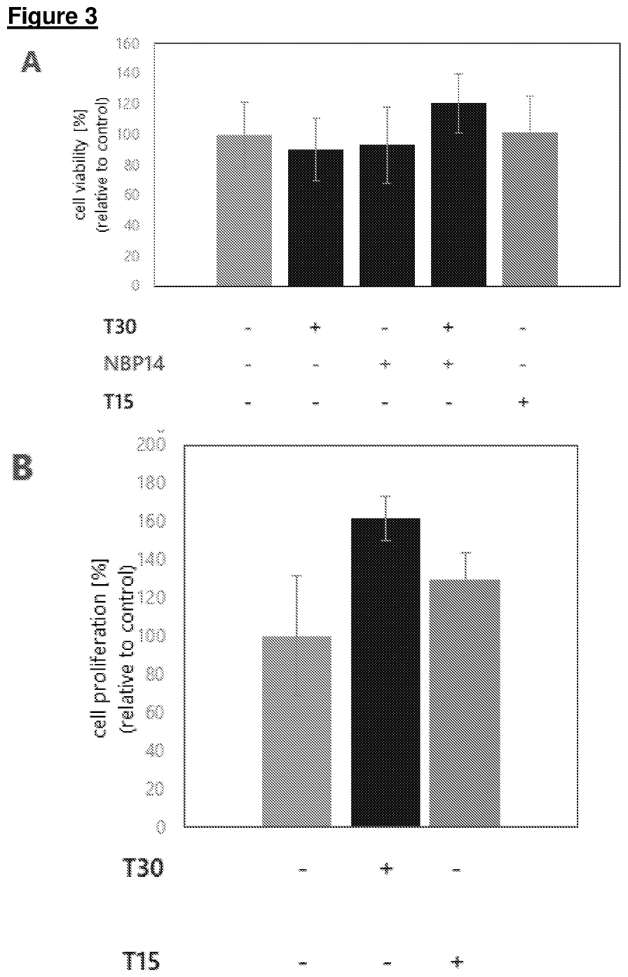

FIG. 2 shows the ability of the cyclic peptide (NBP-14) to inhibit T30-induced intracellular calcium influx in keratinocytes.

FIG. 3 shows A) the degree of cell viability in keratinocyte cell line treated with the linear peptides T30 and T15 and the cyclic peptide NBP-14; and B) cell proliferation of a keratinocyte cell line treated with the linear peptides T30 and T15.

### EXAMPLES

### Rationale

The inventors have generated a number of linear and cyclic peptides based on the C-terminus of acetylcholinesterase known as T15, T30 and NBP-14 peptides and evaluated their effects in a keratinocyte cell line. It should be noted that SEQ ID No: 3 is referred to herein as “cyclised T14”, “CT14” or “NBP-14”, and is a cyclic peptide with an amino acid sequence derived from the C-terminus of Tailed acetylcholinesterase.

### Materials and Methods

### Cyclisation of Peptides

Three techniques were used to achieve cyclization of linear peptides described herein, i.e. side-chain-to-side-chain, side-chain-to-backbone, and head-to-tail (C-terminus to N-terminus) cyclization. Head-to-tail cyclization has been investigated extensively, and can involve directed Cys-Cys disulphide cyclization (up to two per molecule). Careful monitoring of the reaction ensures 100% cyclization. Two general approaches are used for synthesis: (1) classical solution-phase linear peptide cyclization under high dilution conditions; and (2) resin-based cyclization. Two distinct protocols were employed in the solid phase synthesis (1):—
(a) The on-resin cyclization of a peptide anchored via a side-chain functional group, such as imidazole, 3 acid, 4 amine′ or alcohol, was carried out. The peptide was orthogonally protected as an ester at the C-terminus, and the peptide was then assembled through regular Boc or Fmoc synthesis followed by saponification, cyclization and cleavage.

(b) Another protocol that was used was the cyclization cleavage approach, in which the cyclic peptide was synthesized by cyclization after step-wise linear peptide synthesis. One advantage of this method is that the side-chain does not need to be anchored, making the approach more general than (a). (Christopher J. White and Andrei K. Yudin (2011) Nature Chemistry 3; Valero et al (1999) J Peptide Res. 53, 76-67; Lihu Yang and Greg Morriello (1999) Tetrahedron Letters 40, 8197-8200; Parvesh Wadhwani et al (2006) J. Org. Chem. 71, 55-61).

### Example 1—Cyclic T14 (i.e. “NBP-14”)

The ‘tailed’ acetylcholinesterase (T-AChE) is expressed at synapses and the inventors have previously identified two peptides that could be cleaved from its C-terminus, one referred to as “T14” (14 amino acids long), within the other which is known as “T30” (30 amino acids long). The amino acid sequence of the linear peptide, T14, is AEFHRWSSYMVHWK [SEQ ID No:3]. The amino acid sequence of the linear peptide, T30, is KAEFHRWSSYMVHWKNQFDHYSKQDRCSDL [SEQ ID No:2]. Another peptide referred to as “T15” corresponds to the last 15 amino acid residues of SEQ ID No:1, i.e. NQFDHYSKQDRCSDL [SEQ ID No: 4].

The AChE C-terminal peptide “T14” has been identified as being the salient part of the AChE molecule responsible for its range of non-hydrolytic actions. The synthetic 14 amino acids peptide analogue (i.e. “T14”), and subsequently the larger, more stable, and more potent amino acid sequence in which it is embedded (i.e. “T30”) display actions comparable to those reported for ‘non-cholinergic’ AChE.

Referring first to FIG. 1A, there is shown the 14 amino acid long cyclic T14 peptide (i.e. “NBP-14”). The cyclic peptide, NBP-14, has been cyclised via the terminal Alanine (A) and Lysine (K) residues, and is shown in FIG. 1B. Cyclisation can be achieved by several different means. For example, Genosphere Biotechnologies (France) performed the cyclisation of T14 by transforming the linear peptide into an N-terminal to C-terminal lactam. Cyclisation of T14 to create cyclic NBP-14 brings together both ends, i.e. HWK-AEF.

### Example 2—the Effect of Acetylcholinesterase-Derived Peptides on Intracellular Calcium in the Keratinocyte Cell Line HaCaTs Cell Line

The inventors examined the ability of the acetylcholinesterase-derived linear peptides, T30 and T15, and the cyclic peptide NBP-14, to induce intracellular calcium influx into the keratinocyte cell line, and the results are shown in FIG. 2. The application of T30 induced an increase in intracellular calcium in the keratinocyte cell line, while application of the inert T15 peptide did not induce a raise of intracellular calcium in the cell line. NBP-14 was surprisingly able to significantly inhibit the effects of T30.

### Example 3—the Effect of Acetylcholinesterase-Derived Peptides on Proliferation of the Keratinocyte Cell Line HaCaTs Cell Line

The inventors next examined the ability of the acetylcholinesterase-derived peptides, T30 and T15, to induce proliferation of the HaCaT cell line and also tested cell viability to determine cell toxicity of NBP-14, T15 and T30 to the HaCaT cell line. The results are shown in FIG. 3. Referring to FIG. 3B, T30 significantly induced proliferation of HaCaT cells when compared to untreated controls (no peptide). In contrast, T15 did not alter cell proliferation. NBP-14, T15 and T30 do not show induced significant cytotoxic effects in the cell line, as shown in FIG. 3A.

### CONCLUSIONS

The inventors investigated the effects of a cyclic peptide derived from the C-terminus of acetylcholinesterase on a keratinocyte cell line and found that T30, a sequence comprising the T14 sequence, stimulates intracellular calcium influx into the skin cells and induces cell proliferation. The inventors also showed that a cyclic peptide derived from the C-terminus of acetylcholinesterase (known as “NBP-14”) inhibits T30-induced intracellular calcium influx into keratinocytes, but is non-toxic. Accordingly, the inventors believe that NBP-14 can be utilized as a therapeutic agent to treat, prevent or ameliorate skin conditions associated with cell proliferation, such as psoriasis and cancer (e.g. melanoma) and also be used to prevent, reduce and inhibit scar formation.

1. A cyclic polypeptide, derivative or analogue thereof comprising an amino acid sequence derived from the C-terminus of acetylcholinesterase (AChE), or a truncation thereof, for use in treating, preventing or ameliorating a skin disease.

2. The cyclic polypeptide, derivative or analogue thereof, for use according to claim 1, wherein the skin condition which is treated is a skin condition associated with abnormal keratinocyte proliferation.

3. The cyclic polypeptide, derivative or analogue thereof, for use according to either claim 1 or claim 2, wherein the skin condition, which is treated, is selected from a group consisting of: eczema, psoriasis, melanoma, dermatitis, and acne.

4. The cyclic polypeptide, derivative or analogue thereof, for use according to any one of claims 1 to 3, wherein the acetylcholinesterase comprises an amino acid sequence substantially as set out in SEQ ID No:1 or a variant or fragment thereof.

5. The cyclic polypeptide, derivative or analogue thereof, for use according to any one of claims 1 to 4, wherein the cyclic polypeptide, derivative or analogue thereof, comprises:
(i) between 4 and 50 amino acid residues, or between 8 and 40 amino acids; or
(ii) between 6 and 20 amino acids, or between 6 and 15 amino acids.

6. The cyclic polypeptide, derivative or analogue thereof, for use according to any one of claims 1 to 5, wherein the cyclic polypeptide, derivative or analogue thereof comprises cyclic SEQ ID No:2, or a functional variant or fragment thereof.

7. The cyclic polypeptide, derivative or analogue thereof, for use according to any one of claims 1 to 5, wherein the cyclic polypeptide, derivative or analogue thereof comprises cyclic SEQ ID No:3, or a functional variant or fragment thereof.

8. The cyclic polypeptide, derivative or analogue thereof, for use according to any one of claims 1 to 5, wherein the cyclic polypeptide, derivative or analogue thereof comprises cyclic SEQ ID No:4, or a functional variant or fragment thereof.

9. The cyclic polypeptide, derivative or analogue thereof, for use according to any preceding claim, wherein the polypeptide, derivative or analogue thereof is created using a de novo peptide synthesis method.

10. A skin condition treatment pharmaceutical composition comprising a therapeutically effective amount of the cyclic polypeptide, derivative or analogue thereof according to any one of claims 1 to 9, and a pharmaceutically acceptable vehicle.

11. A process for making the skin condition treatment composition according to claim 10, the process comprising combining a therapeutically effective amount of the cyclic polypeptide, derivative or analogue thereof according to any one of claims 1 to 9, with a pharmaceutically acceptable vehicle.

12. A cyclic polypeptide, derivative or analogue thereof comprising an amino acid sequence derived from the C-terminus of acetylcholinesterase (AChE), or a truncation thereof, for use in reducing, preventing or inhibiting scar formation.

13. A cyclic polypeptide, derivative or analogue thereof, for use according to claim 12, wherein the cyclic polypeptide, derivative or analogue thereof is as defined in any one of claims 1 to 9.

14. A method for cosmetic treatment of skin, comprising applying, to skin, a cyclic polypeptide, or a derivative or analogue thereof comprising an amino acid sequence derived from the C-terminus of acetylcholinesterase (AChE), or a truncation thereof.

15. The method according to claim 14, wherein the cyclic polypeptide, derivative or analogue thereof is as defined in any one of claims 1 to 9.

16. The use of a cyclic polypeptide, or a derivative or analogue thereof comprising an amino acid sequence derived from the C-terminus of acetylcholinesterase (AChE), or a truncation thereof, for cosmetic treatment of skin.

17. The use according to claim 16, wherein the cyclic polypeptide, derivative or analogue thereof is as defined in any one of claims 1 to 9.

18. A cosmetic composition comprising a cosmetically effective amount of a cyclic polypeptide, derivative or analogue thereof according to any one of claims 1 to 9, and a cosmetically acceptable vehicle.

19. A process for making the cosmetic composition according to claim 18, the process comprising combining a cosmetically effective amount of the cyclic polypeptide, derivative or analogue thereof according to any one of claims 1 to 1, with a cosmetically acceptable vehicle.

20. The use of the cyclic polypeptide, derivative or analogue thereof according to any one of claims 1 to 9, as a skin whitening agent.


SEQUENCE LISTING (4 sequences)

SEQ ID NO: 1 - protein, 614 aa, Homo sapiens
MRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATDTLDEAERQWKAEFHRWSSYMVHWKNQFDHYSKQDRCSDL

SEQ ID NO: 2 - protein, 30 aa, Homo sapiens
KAEFHRWSSYMVHWKNQFDHYSKQDRCSDL

SEQ ID NO: 3 - protein, 14 aa, Homo sapiens
AEFHRWSSYMVHWK

SEQ ID NO: 4 - protein, 15 aa, Homo sapiens
NQFDHYSKQDRCSDL